(19) United States
(12) Patent Application Publication
(10) Pub. No.: US 2026/0174700 A1
(43) Pub. Date: Jun. 25, 2026

(54) A FORMULATION FOR ORAL DELIVERY OF PEPTIDES AND BIOSIMILARS FOR SYSTEMIC ABSORPTION AND METHOD OF MANUFACTURING THE SAME

(71) Applicant: NBI BIOSCIENCES PVT LTD, Gurugram (IN)

(72) Inventors: Atul KUMAR, Gurugram (IN); Anjali KUMAR, Gurugram (IN); Braj Gaurav SHARMA, Gurugram (IN)

(21) Appl. No.: 19/121,121

(22) PCT Filed: Oct. 13, 2023
(86) PCT No.: PCT/IN2023/050939

§ 371 Date: Apr. 14, 2025

(30) Foreign Application Priority Data
Oct. 13, 2022 (IN) 202211058545

(51) Int. Cl. A61K 9/20; A61K 9/00; A61K 38/00; A61K 47/12

(52) U.S. Cl. CPC A61K 9/2054; A61K 9/0019; A61K 9/0053; A61K 38/00; A61K 47/12; A61K 2300/00

(57) ABSTRACT

The invention relates to formulation for oral delivery of peptides and/or drug molecules and a method of manufacturing the same. The oral formulation is useful for delivering peptides to the gastrointestinal tract, preferably at the ileo-caecal junction of colon.

### FIELD OF THE INVENTION

The invention relates to formulation for oral delivery of peptides and/or drug molecules and a method of manufacturing the same. The oral formulation is useful for delivering peptides to the gastrointestinal tract, preferably at the ileo-caecal junction of colon.

### BACKGROUND OF THE INVENTION

The development of new drugs for the treatment of various diseases has been progressing in recent times, but their use is often restricted because inefficient oral administration. This may be due to the poor oral tolerance, degradation of drug compounds in the stomach, and poor, slow or uneven absorption of the active pharmaceutical ingredient (API) drug. Traditional alternative drug delivery methods, such as intravenous and intramuscular delivery have been useful to deliver therapeutically effective amount of drug into the blood stream or target tissue, however, it is not convenient to patients because of the pain and the risk of infection from needle. Further, the need to use proper sterilization techniques for the use of the drug requires proper medical supervision in patients.

Further, delivery of effective amounts of proteins, peptides and useful enzymes or hormones to prevent or treat a target disease has been problematic. A number of factors are involved in delivering the right amount of the active agent (proteins, peptides and useful enzymes or hormones) in an appropriate drug composition or vaccine delivery system so that it can reach its target site(s) of action for achieving the desired prophylactic or therapeutically effective response.

One of the well-known biological molecules utilized for the regulation of the blood glucose level of a diabetic patient (a disease state in which the pancreas does not release insulin at levels capable of controlling glucose levels) is the hormone Insulin.

The International Diabetes Federation calculated that 366 million people were suffering with diabetes in 2011 and it is expected rise to 552 million by 2030. Though type 2 diabetes mellitus (T2DM) accounts for 85-95% of diabetes, the prevalence of type 1 diabetes mellitus (T1DM) has increased by 2-3% in certain parts of the world, specifically India, Europe and USA. Thus, diabetes has become one of the most common noncommunicable diseases worldwide. All Type-I diabetic patients require Insulin injection throughout their life while more than 50% Type-II patients are on Insulin treatment.

Insulin is a peptide secreted by human glands (such as beta cells of the pancreatic islets) and is considered to be the primary anabolic hormone of the human body and used for the management of sugar metabolism. The molecular formula of human insulin is C257H383N65077S6, which is contained a combination of two peptide chains (dimer) i.e., A-chain (21 amino acid) and B-chain (30 amino acid), which are linked together by two disulphide bonds. Insulin is widely used for the treatment of all Type-I and severe Type-II diabetic patients.

At present, insulin is primarily administered through various routes such as intravenously, intramuscularly, subcutaneous injection only. However, there are problems with the existing injectable Insulin delivery systems (IDS) such as hypoglycaemia risk, skin allergies such as lipoatrophy, lypohypertrophy, painful procedure, inconvenience to carry, injection/needle pricking associated infection etc.

Further, existing oral delivery of peptides involves problems such as degradation of peptide by stomach acids, degradation of peptide by the intestinal protease & peptidase enzymes, pre-mature delivery of Insulin, poor bioavailability in the gastrointestinal (GI) tract because of tight junction, mucus layer, uneven dose uniformity etc.

Furthermore, factors that is required for effective diabetes treatment is that Insulin needs to remain in circulation in the blood to control the amount of glucose. The other route of Insulin delivery such as oral, rectal, and nasal has never achieved considerable success toward systemic action of a peptide such as Insulin.

In addition, combination of Insulin along with other drug molecules is required in some instances to control the symptoms of diabetes. Accordingly, oral formulations are required which can deliver distinctively & separately or in combination a drug without its release at stomach for the class of drugs need delayed and sustained release in the small intestinal area i.e. duodenum & jejunum as well as in Colon.

Thus, there is a need for an improved oral formulation for delivering a delivery of peptides, such as insulin and/or drug molecules to the small intestine and colon, without premature delivery of the product to the upper gastrointestinal (GI) tract and also ameliorating at least one of the aforementioned drawbacks.

### SUMMARY OF THE INVENTION

In one aspect, the present invention provides an oral formulation comprising: peptides or biosimilar; alginates or an alkali salt thereof, at least one polymer, crosslinker; and one or more pharmaceutically acceptable excipients, to the systemic circulation while preventing disintegration from stomach acid and intestinal enzymes.

In another aspect, the present invention provides an oral peptide delivery formulation in combination with at least one drug.

In another aspect, the present invention provides a method for preparing the oral delivery formulation.

In yet another aspect, the present invention provides an oral formulation of polymerized peptides for use in the oral delivery formulation and a method preparing the same.

In yet another aspect, the present invention provides a polymerized Insulin for use in the oral delivery formulation and a method preparing the same.

In still another aspect, the present invention provides a method for the treatment of a patient by delivering peptides and/or drug molecules to a predetermined location in the GI tract.

In yet another aspect, the present invention relates to the use of the oral drug delivery formulation for delivering peptides and/or drug molecules to a predetermined location in the gastrointestinal tract or for delivering diagnostic aids/agents.

### BRIEF DESCRIPTION OF THE DRAWINGS

Reference will be made to embodiments of the invention, examples of which may be illustrated in accompanying figures. These figures are intended to be illustrative, not limiting. Although the invention is generally described in the context of these embodiments, it should be understood that it is not intended to limit the scope of the invention to these particular embodiments.

FIG. 1A shows the FT-TR spectrum of insulin glargine

FIG. 1B shows the FT-IR spectrum of insulin glargine loaded nanoparticles

FIG. 1C shows the FT-IR spectrum of physical mixture of insulin glargine loaded nanoparticle and HPMC K 100

FIG. 1D shows the FT-IR spectrum of physical mixture of insulin glargine loaded nanoparticle and gaur gum

FIG. 1E shows the FT-IR spectrum of physical mixture of insulin glargine loaded nanoparticle and Aerosil

FIG. 1F shows the FT-IR spectrum of physical mixture of insulin glargine loaded nanoparticle and Avicel PH 101

FIG. 1G shows the FT-IR spectrum of physical mixture of insulin glargine loaded nanoparticle and magnesium stearate

FIG. 1H shows the FT-IR spectrum of physical mixture of insulin glargine loaded nanoparticle and all excipients.

FIG. 2A shows the DSC thermogram of insulin glargine.

FIG. 2B shows the DSC thermogram of insulin glargine loaded nanoparticle.

FIG. 2C shows the DSC thermogram of physical mixture of insulin glargine loaded nanoparticle.

FIG. 2D shows the DSC thermogram of physical mixture of insulin glargine loaded nanoparticle and Guar gum

FIG. 2E shows the DSC thermogram of physical mixture of insulin glargine loaded nanoparticle and Aerosil.

FIG. 2F shows the DSC thermogram of physical mixture of insulin glargine loaded nanoparticle and Avicel PH101.

FIG. 2G shows the DSC thermogram of physical mixture of insulin glargine loaded nanoparticle and magnesium stearate.

FIG. 2H shows the DSC thermogram of physical mixture of insulin glargine loaded nanoparticle and all excipients.

FIG. 3 shows Insulin Glargine concentration

FIG. 4 shows Graphical representation of percentage uptake of insulin by cells treated with test samples N17A, N17B and N17C and N18 Placebo

### DESCRIPTION OF THE INVENTION

In one aspect, the present invention relates to a microbial-triggered oral intestinal delivery formulation. The human intestine harbors higher microbial flora, which has been targeted in developing the microbial-triggered oral intestinal delivery formulation of the present invention. The formulation of the present invention delivers the peptides and/or drug molecules to a predetermined location in the GI tract.

Accordingly, the present invention is directed towards an oral delivery formulation comprising peptides orbiosimulars and/or drug molecules, plant extract or their derivatives and one or more pharmaceutically acceptable excipients.

As used herein, the phrase “peptides” refers to those compounds or materials which function as an active pharmaceutical ingredient (API) for veterinary use as well as human pharmaceutical use. In a preferred embodiment, the “peptides” refers to, but not limited to, hormones, vaccines etc.

In a preferred embodiment, the “peptides” refers to, but not limited to, Insulin, calcitonin, ACTH, glucagon, somatostatin, somatotropin, somatomedin, parathyroid hormone, erythropoietin, hypothalamic releasing factors, prolactin, thyroid stimulating hormones, endorphins, enkephalins, vasopressin, non-naturally occurring opioids, superoxide dismutase, interferon, asparaginase, arginase, arginine deaminease, adenosine deaminase, ribonuclease, trypsin, chymotrypsin, papain, Ara-A (Arabinofuranosyladenine), Acylguanosine, Nordeoxyguanosine, Azidothymidine, Didesoxyadenosine, Dideoxycytidine, Dideoxyinosine Floxuridine, 6-Mercaptopurine, Doxorubicin, Daunorubicin, or I-darubicin, Erythromycin, Vancomycin, oleandomycin, Ampicillin; Quinidine and Heparin proteins, peptides, nucleosides, nucleotides, antiviral agents, antineoplastic agents, antibiotics, antiarrhythmics, anti-coagulants, Monoclonal antibody, vaccines, etc.

In one aspect of the invention wherein central core comprises the polymer selected from poly-g-glutamic acid, Starch, Polyethylene glycol, chitosan, alginates.

In one aspect of the invention plant extract or their derivatives is selected from galactomannan, polysaccharide, chitosan, gaur gum, gaur Arabic, gum acacia and fenugreek extracted powder or their combination. In preferred aspect of the invention plant extract or their derivatives is selected from gaur gum.

In one aspect of the invention disintegration agent preferably selected from, micro crystalline cellulose (MCC), cross linked PVP, Cellulose, PVP, starch, alginic acid and calcium silicates etc or their combination.

In another aspect of the invention, primary coating further comprises protease inhibitor. In one aspect, protease inhibitor is selected from Potato, Serpine, Cereal, Rapeseed, Mustard, Squash, Fenugreek, Legume, or combination thereof.

In another aspect of the invention, primary coating further comprises hydroxypropyl methylcellulose (HPMC).

As used herein, the phrase “Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

The formulation of the disclosure is formulated to be compatible with its intended oral route of administration. Accordingly, the excipient can be any excipient known in the art.

In a preferred embodiment, the excipient is selected from the list consisting of, but not limited to, Magnesium Stearate, Talc, Silicone dioxide, Stearic acid, Sodium starch glycolate, Gelatin, Corn starch, Polyethylene glycol, and Starch.

In preferred aspect of the invention, peptide is natural or synthetic insulin. In one aspect Insulin glargine is used, which is a synthetic version of human insulin to treat adults and children with type 1 diabetes and adults with type 2 diabetes to improve and maintain glycemic control.

In one aspect of the invention secondary coating of the tablet comprises plasticizers.

In another aspect of the invention, the secondary coating comprises of Opadry white y-1-700/other Opadry.

In another aspect of the invention, outer coating is selected from Eudragit-L-100 or Eudragit S-100 or fenugreek extract or HPMC-P a combination thereof.

In another aspect of the invention, outer coating is selected from Eudragit-L-100 or Eudragit S-100 or fenugreek extract or HPMC-P a combination thereof preferably in the range of from 10 wt % to 90 wt %.

In an aspect of the present invention, an oral formulation comprises: peptides or biosimilar; alginates or an alkali salt thereof; at least one polymer; a crosslinker; and one or more pharmaceutically acceptable excipients, wherein at least one polymer is selected from arabinoxylans, gelatin, pectin, guar gum, hyaluronic acid, mucin, poly-amino acids, chitosan, dextran, PEG, galactomannan, starch.

In a preferred embodiment, the oral formulation is a nanoparticle.

In a preferred embodiment, the peptide in oral formulation the peptide is selected from insulin, preferably human insulin, Long-acting, Short-acting, Rapid-acting, Intermediate-acting and Mixed Insulin.

In a preferred embodiment, the insulin is insulin glargine

In a preferred embodiment, the alginates or an alkali salt thereof is selected from sodium alginate

In a preferred embodiment, the chitosan has molecular weight range of 20 kda to 2000 kda.

In a preferred embodiment, the chitosan is more than 85% deacylated chitosan having molecular weight in the range of 190 Daltons to 310 Daltons.

In a preferred embodiment, the crosslinker is selected from calcium chloride, calcium salicylate, collagen, lignin, carbodiimides or combination thereof.

In a preferred embodiment, the oral formulation is a nanoparticle encapsulating insulin glargine.

In a preferred embodiment, the oral formulation comprises the peptide in the range of 0.1 to 60%.

In a preferred embodiment, the oral formulation comprises the insulin in the range of 1 IU to 200 IU, preferably 20 IU to 110 IU.

In a preferred embodiment, the oral formulation comprises the alginic acid or an alkali salt thereof is in the range of 0.1 wt % to 50 wt %, preferably 1 wt % to 2 wt %.

In a preferred embodiment, the oral formulation comprises the crosslinker is in the range of 0.01% to 10%.

In a preferred embodiment, the oral formulation comprises pharmaceutically acceptable excipients preferably selected from polyvinyl alcohol, polaxmer188, glycerol or derivative thereof, fatty acids, ethylene oxide, alcohol, bile salts, phospholipids or combination thereof.

In a preferred embodiment, the oral formulation optionally comprising at least one drug.

In a preferred embodiment, the oral formulation are preferably selected from pill, powder, capsule, or nano-emulsion.

In another aspect of the present invention relates to a process for preparing an oral formulation comprising the following steps:
preparing insulin solution of different strength in water by adjusting the 4.0 pH,
adding insulin solution to 1% sodium alginate,
adding chitosan in acetic acid to an aqueous solution mixing with continuous homogenizing the solution at 12000 rpm at 2-8° C. for 10 minutes to prepare a first homogenized mixture;
optionally adding an aqueous solution containing a mixture of pharmaceutical excipients to the first homogenized mixture containing insulin and mixing to prepare a second homogenized mixture;
sonicating the second homogenize mixture for 3 mins in an ice bath followed by centrifuging for 60 mins at 10,000 rpm, washing only one time and centrifuging again for 5 mins to prepare the nanoparticle pellets;
freeze drying the pellet to obtain the oral formulation.

In a preferred embodiment of the process, the chitosan in acetic acid is in the ratio of 1:1.

In a preferred embodiment of the process, the sodium alginate in the first homogenized mixture is in the range 0.2% to 10%.

In a preferred embodiment of the process, the insulin solution is in the range 0.1% to 50%.

In a preferred embodiment of the process, the first homogenized mixture was prepared by mixing at 5000 to 15000 rpm, second homogenized mixture was prepared by mixing at 5000 to 15000 rpm.

In a preferred embodiment of the process, the first homogenized mixture was mixed for 5 to 20 minutes, second homogenized mixture was mixed for 2 to 10 minutes. And centrifuge the second homogenize for 60 min at 10,000 rpm and at 2-8° C. temperature.

In yet another embodiment, the process comprises the following steps:
adding chitosan (2% w/v) in acetic acid (1% v/v) to 20 ml of Sodium Alginate as the aqueous phase and homogenizing the mixture for 10 min at 12,000 rpm.
Adding 5 ml of insulin solution (100 IU/ml) to the mixture and mixing it gently using a magnetic stirrer for 3 min at 200 rpm.
adding an aqueous phase containing a mixture of 5 ml of Poloxamer-188 2 and 5 ml of PVA to the phase containing the insulin and chitosan and homogenizing for 5 min at 12000 rpm
Sonicating the preparation at 80 W for 2 min in an ice bath followed by centrifuging (10,000 rpm, 10° C., 60 min) and after two washing it was centrifuged again at 10,000 rpm for 15 min;
Freeze-drying to obtain encapsulated Insulin glargine.

In another aspect of the present invention relates to a method for treatment of diseases by administering the oral formulation according to the present disclosure.

In a preferred embodiment, the oral formulation is administered for decreasing blood glucose or for treating conditions related to elevated blood glucose in a human comprising administering the oral formulation as claimed in claim 1, wherein the condition related to elevated blood glucose is selected from the group consisting of Type 1 diabetes, Type 2 diabetes, impaired glucose tolerance, hyperglycemia, insulin resistance syndrome, and glucosuria.

In a preferred embodiment, the oral formulation is administered for delivering peptides to the gastrointestinal tract, preferably insulin at the ileo-caecal junction of colon.

In a preferred embodiment, the peptides are selected from Bortezomib, Cosyntropin, Saralasin, Vancomycin, Icatibant, Salmon calcitonin, Pentagastrin, Corticorelin, Cyclosporin, Urofollitropin, Glucagon, Exenatide, Teduglutide, Eptifibatide, Buserelin, Enfuvirtide, Somatorelin, Linaclotide, Romidepsin, Vapreotide, Ziconotide, Mifamurtide, Carbetocin, Teriparatide, Secretin (human), Somatostatin, Bortezomib, Cosyntropin, 26S proteasome Bortezomib ACTH er Cosyntropin Angiotensin II, Bradykinin B2, Corticorelin or combination thereof.

Yet another aspect of the present invention relates to use of the oral formulation for the treatment of diseases selected from cortisol disorder, Anti-hypertension, Hereditary angioedema, osteoporosis, immunosuppressant, fertility treatment, irritable bowel syndrome, myocardial infarction, Sex hormone-responsive cancers, anti-HIV, hormone deficiency, irritable bowel syndrome, esophageal variceal bleeding, chronic pain, osteosarcoma, postpartum bleeding, osteoporosis, multiple myeloma or prostate cancer.

Yet another aspect of the present invention relates to use of the oral formulation for delivering diagnostic aids/agents.

In a preferred embodiment, the peptide is Insulin, preferably a polymerized form. The Insulin-API is converted into the polymerized form in order to provide the pH, moisture and temperature stability and will be significant to maintain the bulk of peptide.

In a preferred embodiment, the polymerized insulin is prepared using different methodologies mentioned below:

G-PGA based method: Poly-g-glutamic acid (g-PGA) water-soluble, biodegradable, and non-toxic polymer solution is prepared and Diethylenetriamine pentaacetate (DTPA) is added to the solution in order to prevent insulin from enzymes degradation. Retention of insulin is further enhanced by adding MgSO4 during polymerization. Chitosan may also be added to improve the quantity of bulk containing Insulin.

Starch-PEG based method: Hydrophobic starch acetate is conjugated with polyethylene glycol (PEG) to form an amphiphilic polymeric derivative. Insulin is added to the solution of Starch-PEG. It forms a pH-sensitive, the Pegylated starch acetate nanoparticles are capable of releasing insulin at junctions of intestine. The bulk quantity of coated insulin may vary according to the starch quantity added to the solution.

Chitosan-alginate based method: Alginate, an anionic mucoadhesive polysaccharide consisting of various ratios of b-D-mannuronopyranosyl and a-L-guluronopyranosyl units linked by (1-4)-O-glycosidic bonds is utilized for preparing microparticles. It shows a property of mild gel-formation by ionically cross-linked with multivalent cations, such as calcium ions, thus enabling drug retention within the gel matrix. The large porosity of alginate beads often leads to low encapsulation efficiency and rapid release of the peptide drug such as Insulin (Low drug encapsulation efficiency of alginates can be improved by the addition of chitosan and dextran sulphate). Highest loading efficiency and remarkable activity maintenance is achieved when the insulin is loaded during the chitosan solidification process, while the network formation between chitosan and alginate-Ca microspheres reduces the porosity and decreases the leakage of insulin.

Calcium alginate bead method: The Insulin is added to the Sodium alginate (1%) solution, mixed and incubated for 5 hours. Then Insulin will be encapsulated by the Sodium alginate. Later, the insulin-loaded sodium alginate solution will be added to the 4% Calcium chloride solution through the extrusion process. The extruded alginate-chloride-insulin bead will be collected and will further processed cold dry using different methodology such as spray drying etc.

The polymerized form of peptide is useful for smooth absorption of peptides from the intestinal walls to systemic circulation by microvilli, colonic mucosa, and mucus lining.

In a preferred embodiment, the microbial-triggered oral drug delivery formulation contains drug molecules.

As used herein the “drug molecules” refers to chemical synthetic drugs such as Alpha-glucosidase inhibitors, metformin, sitagliptin, glipizide, Dipeptidyl peptidase-4 (DPP-4) inhibitors, Glucagon-like peptide-1 receptor agonists (GLP-1 receptor agonists), Meglitinides, Sodium-glucose transporter (SGLT) 2 inhibitors, Sulfonylureas, Thiazolidinediones, Dopamine-bromocriptine, Bisacodyl, Diclofenac sodium, misoprostol, Diltiazem, Aspirin, Erythromycin, Omeprazole, Didanosine and others.

In preferred aspect of the invention, active ingredient or drug is selected from metformin or sitagliptin or combination thereof.

In a preferred embodiment, the microbial-triggered oral drug delivery formulation is capable of releasing a small portion of the drug molecules in the small intestine & further release of peptide, biosimilar, or Insulin at the action site (ileo-caecal junction/Colon).

The combined release of active-pharmaceutical ingredients (API) i.e., peptides and biosimilars provides better, enhanced, and prolonged release of API, resulting in great relief to the patient by reducing the dosage concentration, frequency, and side effects of the API.

In a preferred embodiment, the oral drug delivery formulation comprises multiple coatings by the natural extracted polymer such as polysaccharide/fenugreek extract/guar gum and then upper coatings that support the formulation for its non-sticky property and enhanced transit inside the GI tract.

In a preferred embodiment, granulation of polymerized peptide is done with fenugreek extracted D2O water.

In a preferred embodiment, the core tablet is prepared using a formulation comprising the polymerized peptide/insulin granules and/or biosimilars and/or other drugs such as metformin, sitagliptin etc. Formulation will be done by wet granulation or direct compression technique and compression with optimized set of upper punch & lower punch.

In a preferred embodiment, the core tablet will be coated by a secondary coating of plasticizers (Seal coating).

In a preferred embodiment, final coating material is selected from the combination of Eudragit-L-100, Eudragit S-100, HPMC-P, Fenugreek extract and others.

In a preferred embodiment, the method for preparing the oral peptide and/or biosimilar drug delivery formulation comprising the following steps:
1. Sifting: Co-Sift API powder i.e., Insulin/Peptide/Biosimilar, Guar gum/Gum Arabic/Fenugreek extracted powder/Gum acacia/Plant based polysaccharide/Plan based galactomannans etc or in combination and Avicel through #30 sieve using vibratory sifter and collect in polybag. Mix all the material for 10 minutes.
2. Pre-coat solution prepare: Dissolve Polyvinyl Pyrrolidone K90 (PVP K90) IP in Purified Water under mechanical stirring until PVP K90 is completely dissolved, and solution is formed or use the fenugreek extracted water in different ratio like 1:1 to 1:10 explain in the methodology to develop the initial coating and binder material.
3. Granulation: Transfer dry mix sifted materials from step 1 into rapid mixer granulator. Add complete binder solution of step 2 at slow rate into the dry mix at slow impeller speed and chopper off setting. Additionally, if required mix the granules for approximately at slow impeller and chopper speed to get desired consistency of granules.
4. Drying: Dry the wet granules at inlet temperature in a range from 20° C. to 45° C. in Fluidized Bed Dryer (FBD). Rake the granules. Check the loss on drying (LOD) of the granules, once LOD reached up to 6-7% w/w remove the semi-dried granule from FDB and wet mill the granules.
5. Wet Milling: Mill the wet granules through a 2 mm screen at 2100 RPM (Knive reverse setting) using Multi Mill.
6. Drying: Continue the drying at inlet temperature in a range from 20° C. to 45° C. in FBD to achieve the target LOD approximately 2.5 to 3.5% w/w. (Limit: NMT 5.0% w/w).
7. Sifting and Milling: Mill the dried granules using multi mill at 2100 RPM (Knive reverse setting) through 1 mm screen. Sift milled granules through #30 sieve using vibratory sifter. Mill the retained granules by using multi mill at 2100 RPM (Knive reverse setting) through 1 mm screen and collect in polybag. Sift milled granules through #30 sieve using vibratory sifter. If retentions remain, repeat the step of sifting and milling.
8. Dried granules and extra granular material to be taken for blending: If yield is less than 98% of the dried granules, then calculate the extra granular materials according to the corresponding yield. If the yield of dried granules is more than or equal to 98.0%, dispense the theoretical quantity of extra granular materials.
9. Sifting of Extra Granular Materials: Co-Sift the talc and Colloidal Silicon Dioxide IP through #40 sieve using vibratory sifter and collect in polybag. Sift Magnesium Stearate IP through #60 sieve using vibratory sifter and collect in polybag.
10. Blending: Load the dried granules of step 8 into blender, add sifted material of step 9 and mix for 10 minutes at 15 RPM.
11. Lubrication: Add sifted material of step 10 and mix for 5 minutes at 15 RPM.
12. Compression: Compress the lubricated blend of step 11 using Bi-layer compression machine with the following in-process control checks.
13. Seal Coating-Preparation of coating dispersion: Take purified water in S.S vessel. Add Opadry White y-1-700 slowly under continuous stirring to avoid lump formation. After addition of all the Opadry White y-1-700/other Opadry, reduce the mixing speed to nearly eliminate the vortex. Mix for at least 45 minutes to form uniform dispersion.
14. Seal Coating-Coating process: Load the de-dusted & inspected core tablets into a clean, dry S.S. coating pan. Set the standard parameters as provided in the record. Before starting the coating process, maintain the bed temperature to 10±5° C.-60±5° C. Record the weight of 100 Tablets to find the average weight of tablet. Apply the film coating solution to the tablets using a clean spray gun assembly to get 1% to 20% w/w. Coating dispersion to be stirred continuously throughout the coating process at RPM 20 to 500. Checking the inlet & exhaust temperature, spray rate, bed temperature at every 10-min.
15. Final coating-Preparation of coating dispersion: Take purified water and isopropyl alcohol in 1:2 to 1:9 ratio in S.S vessel. Add Eudragit L100/S100/Fenugreek extracted solution from 10 wt % to 90 wt %, slowly under continuous stirring to avoid lump formation. After addition of Tri-ethyl citrate and talc to the solution from step 3.5.3 by continuous stirring. The vortex, sonication, homogenization required for the proper mixing of ingredients.
16. Final coating-Coating process: Load the de-dusted & inspected core tablets into a clean, dry S.S. coating pan. Set the standard parameters as provided in the record. Before starting the coating process, maintain the bed temperature to 10-70±5° C. Record the weight of 100 Tablets to find average weight of tablet. Apply the coating solution to the tablets using a clean spray gun assembly to get 1%-20% w/w. Coating dispersion to be stirred continuously throughout the coating process at RPM 10 to 500. Check the inlet & exhaust temperature, spray rate, bed temperature at every 10-min.

In a preferred embodiment, the method for preparing the oral peptide and biosimilar drug delivery formulation comprising the following coating parameters:

| Sr. No | Parameters | Limits |
|---|---|---|
| 1 | Net weight of core Tablet | 20 mg to 1400 mg |
| 2 | Spray gun nozzle diameter | 0.1 mm to 1.2 mm |
| 3 | Gun to tablet bed distance | 2 cm to 100 cm |
| 4 | Inlet temperature | 10° C.-80° C. |
| 5 | Exhaust temperature | 5° C.-70° C. |
| 6 | Bed temperature | 5° C.-70° C. |
| 7 | Atomizing air pressure (Bar) | 1.0-5.0 BAR |

In a preferred embodiment, the method for preparing the oral peptide and biosimilar drug delivery formulation comprising the following in process checks during coating:

| Sr. No | Parameters | Limits |
|---|---|---|
| 1 | Appearance | Convex oval shape |
| | | of 8 mm diameter |
| 2 | Average weight of 10 | ±2%-15% |
| | enteric coated tablet | |
| 3 | Thickness (Average of | ±0.1 mm- |
| | 10 tablet) | 3.0 mm |

In a preferred embodiment, the commensals or bacteria available in the small intestine and playing an important role in release of the peptide and the biosimilar is selected from Alcaligenes faecalis, Bacteroides spp, Bacteroides fragilis, Clostridium spp, Clostridium sordellii, Enterococcus spp, Enterococcus faecalis, Enterococcus faecium, Escherichia coli, Actinomyces spp, Aeromonas spp, Aggregatibacter actinomycetemcomitans, Achromobacter spp, Streptococcus viridans, Vibrio spp, Eubacterium spp, Faecalibacterium spp, Flavobacterium spp, Lactobacillus spp, Methanobrevibacter smithii Intestines, Mycobacteria spp, Mycoplasma spp, Pseudomonas aeruginosa, Ruminococcus bromii, Sarcina spp, Staphylococcus aureus, Staphylococcus epidermidis, Streptococcus anginosus.

In a preferred embodiment, the commensals or bacteria available in the large intestine and playing an important role in release of the peptide and the biosimilar is selected from Achromobacter spp, Acidaminococcus fermentans, Acinetobacter calcoaceticu, Actinomyces spp, Aeromonas spp, Anaerobiospirillum spp, Alcaligenes faecalis, Bacillus spp, Bacteroides spp, Bacteroides fragilis, Bacteroides melaninogenicus, Bacterionema matruchotii, Bifidobacterium spp, Plesiomonas shigelloides, Yersinia enterocolitica, Escherichia coli, Eubacterium spp, Faecalibacterium spp, Flavobacterium spp, Fusobacterium spp, Morganella morganii, Mycobacteria spp, Mycoplasma spp, Peptococcus spp, Peptostreptococcus spp, Sarcina spp, Staphylococcus aureus, Streptococcus anginosus, Streptococcus viridans, Veillonella spp, Vibrio spp, Butyriviberio fibrosolvens, Campylobacter spp, Campylobacter coli, Clostridium spp, Clostridium difficile, Clostridium sordellii, Cutibacterium acnes, Pseudomonas aeruginosa, Ruminococcus bromii, Eikenella corrodens, Enterobacter cloacae, Enterococcus spp, Enterococcus faecalis, Enterococcus faecium, Propionibacterium spp, Ruminococcus spp.

In a preferred embodiment, the oral peptide and biosimilar drug delivery formulation according to the present disclosure can deliver more than 90% of peptides and/or API/biosimilars to the active site for systemic absorption such as ileo-caecal junction or large intestine.

In a preferred embodiment, the oral drug delivery formulation will deliver the peptides and/or API/biosimilars once the different intestinal bacteria mentioned above will interact with the orally ingested tablet.

In a preferred embodiment, the combined release of peptides and/or API/biosimilars is to provide the better, enhanced, prolonged release of peptides and/or API/biosimilars resulting in great relief to the patient by reducing the dosing concentration, frequency, and side effects.

In a preferred embodiment, the oral drug delivery formulation will deliver insulin to the colon (>95%) without its release at upper part of GI tract i.e., stomach and small intestine. Accordingly, Insulin may be prevented from the degradation by stomach acid and intestinal enzyme and thus oral drug delivery is possible through present approach.

In a preferred embodiment, the oral drug delivery formulation will be acted upon by the gut flora of ileo-caecal junction and large intestine and will cause the drug to be released in the large intestine/colon only.

In a preferred embodiment, the drug molecules which are targeted to the colon can be retained in the colon up to 5 days according to the present formulation. Accordingly, oral drug delivery formulation may be employed in the reduction of dosing frequency of the patient.

The foregoing description of the invention has been set merely to illustrate the invention and is not intended to be limiting. Since the modifications of the disclosed embodiments incorporating the spirit and substance of the invention may occur to the person skilled in the art, the invention should be construed to include everything within the scope of the disclosure.

### EXAMPLES

The above-described aspects and embodiments of the invention further be understood by following non-limiting examples and corresponding drawing figures.

### Example 1: Preparation of Insulin Nanoparticle

Synthesis: 10 ml of chitosan (2% w/v) in acetic acid (1% v/v) was added to 20 ml of Sodium Alginate or Eudragit or PLGA as the aqueous phase. Mixture was homogenized for 10 min at 1 2,000 rpm. 5 ml of insulin solution (100 IU/ml) was added to the mixture and mixed gently using a magnetic stirrer for 3 min at 200 rpm. Aqueous phase containing a mixture of 5 ml of Poloxamer-1882 and 5 ml of PVA was added to the phase containing the insulin and chitosan and homogenized for 5 min at 1 2000 rpm. The preparation was sonicated at 80 W for 2 min in an ice bath. Preparation was centrifuged at 10,000 rpm, 10° C. for 60 min and after washing was centrifuged at 10,000 rpm for 15 min. (washed twice). The preparation was freeze-dried to obtain encapsulated Insulin glargine. The composition of different insulin glargine loaded nanoparticle preparation according to the present disclosure are provided in the Table 1 below:

TABLE 1
Composition of the different insulin glargine loaded nanoparticles

| Ingredients | N01 | N1 | N2 | N3 | N4 | N5 | N6 | N7 | N8 | N9 | N10 | N11 |
|---|---|---|---|---|---|---|---|---|---|---|---|---|
| Insulin (ml) | 5 | 5 | 5 | 5 | 5 | 5 | 5 | 5 | 5 | 5 | 5 | 3 |
| (100 IU/ml) | | | | | | | | | | | | |
| Insulin (ml) | — | — | — | — | — | — | — | — | — | — | — | — |
| (50 IU/ml) | | | | | | | | | | | | |
| Insulin (ml) | — | — | — | — | — | — | — | — | — | — | — | — |
| (25 IU/ml) | | | | | | | | | | | | |
| Sodium | 2 | 1 | 0.1 | 0.2 | 0.5 | — | 0.2 | 0.5 | 0.2 | 0.5 | — | 0.5 |
| Alginate (% | | | | | | | | | | | | |
| w/v) | | | | | | | | | | | | |
| Volume of | 20 | 20 | 20 | 20 | 20 | — | 20 | 20 | 20 | 20 | — | 30 |
| the Sodium | | | | | | | | | | | | |
| Alginate (ml) | | | | | | | | | | | | |
| Eudragit RS | — | — | — | — | — | 20 | — | — | — | — | 20 | — |
| (1% w/v) ml | | | | | | | | | | | | |
| PLGA | — | — | — | — | — | — | — | — | — | — | — | — |
| (% w/v) | | | | | | | | | | | | |
| Polaxmer188 | 5 | 5 | 5 | 5 | 5 | 5 | 5 | 5 | 5 | 5 | 5 | 5 |
| 2% w/v (ml) | | | | | | | | | | | | |
| Chitosan (% | 2 | 2 | 2 | 2 | 2 | 2 | 0.5 | 0.5 | 1 | 1 | 0.5 | 0.5 |
| w/v) | | | | | | | | | | | | |
| Chitosan (ml) | 10 | 10 | 10 | 10 | 10 | 10 | 10 | 10 | 10 | 10 | 10 | 10 |
| Polyvinyl | 5 | 5 | 5 | 5 | 5 | 5 | 5 | 5 | 5 | 5 | 5 | 5 |
| Alcohol Cold | | | | | | | | | | | | |
| 1% w/v (ml) | | | | | | | | | | | | |
| Calcium | — | — | — | — | — | — | — | — | — | — | — | — |
| chloride (500 | | | | | | | | | | | | |
| mM) ml | | | | | | | | | | | | |

| Ingredients | N12 | N14 | N15 | N16 | N17A | N17B | N17C | N18 | N19 | N20 |
|---|---|---|---|---|---|---|---|---|---|---|
| Insulin (ml) | 3 | 3 | 3 | 3 | 15 | — | — | — | — | 15 |
| (100 IU/ml) | | | | | | | | | | |
| Insulin (ml) | — | — | — | — | — | 15 | — | — | — | — |
| (50 IU/ml) | | | | | | | | | | |
| Insulin (ml) | — | — | — | — | — | | 15 | — | — | — |
| (25 IU/ml) | | | | | | | | | | |
| Sodium | 0.5 | 1 | 1 | 1 | 1 | 1 | 1 | 1 | — | — |
| Alginate (% | | | | | | | | | | |
| w/v) | | | | | | | | | | |
| Volume of | 20 | 10 | 20 | 20 | 60 | 60 | 60 | 60 | — | — |
| the Sodium | | | | | | | | | | |
| Alginate (ml) | | | | | | | | | | |
| Eudragit RS | — | — | — | — | — | — | — | — | 60 | — |
| (1% w/v) ml | | | | | | | | | | |
| PLGA | — | — | — | — | — | — | — | — | — | 2 |
| (% w/v) | | | | | | | | | | |
| Polaxmer188 | 5 | 5 | 5 | 5 | 15 | 15 | 15 | 15 | 15 | 5 |
| 2% w/v (ml) | | | | | | | | | | |
| Chitosan (% | 0.5 | 0.5 | 1 | 1 | 1 | 1 | 1 | 1 | 1 | 2 |
| w/v) | | | | | | | | | | |
| Chitosan (ml) | 10 | 10 | 10 | 10 | 30 | 30 | 30 | 30 | 30 | 10 |
| Polyvinyl | 5 | 5 | 5 | 5 | 15 | 15 | 15 | 15 | 15 | 5 |
| Alcohol Cold | | | | | | | | | | |
| 1% w/v (ml) | | | | | | | | | | |
| Calcium | 1 | 1 | 1 | 4 | 3 | 3 | 3 | 3 | 3 | — |
| chloride (500 | | | | | | | | | | |
| mM) ml | | | | | | | | | | |

In composition formulations N6 to N20 insulin was added into the aqueous sodium alginate solution. Furthermore, in these trials the number of washings was reduced to 1 from 2. To the formulations N12 to N16 Calcium chloride (50 millimolar) of different amount was added as cross linker and volume of insulin was reduced to 3 mL from 5 mL.

### Example 2: Insulin Loaded Nanoparticle Formulation Percentage Yield

The prepared insulin nanoparticle loaded formulations prepared were characterized for their percentage yield (Table 2). Percentage yield was calculated using the formula: (Practical amount/Theoretical amount of ingredients)×100

TABLE 2

| S No | Formulation code | Percent yield (%) |
|---|---|---|
| 1 | N01 | 10.569 |
| 2 | N1 | 22.006 |
| 3 | N2 | 0.338 |
| 4 | N3 | 0.443 |
| 5 | N4 | 7.19 |
| 6 | N5 | 1.47 |
| 7 | N6 | 7.19 |
| 8 | N7 | 1.83 |
| 9 | N8 | 7.89 |
| 10 | N9 | 1.79 |
| 11 | N 10 | 12.18 |
| 12 | N 11 | Not lyophilized |
| 13 | N 12 | Not lyophilized |
| 14 | N 14 | Not lyophilized |
| 15 | N 15 | 84.43 |
| 16 | N 16 | 76.08 |
| 17 | N 17 A | 61.673 |
| 18 | N17 B | 95.185 |
| 19 | N17 C | 93.238 |
| 20 | N18 | 94.259 |
| 21 | N19 | 98.276 |
| 22 | N20 | Not formed |

### Example 3: Insulin Loaded Nanoparticle Formulation Free Drug Percentage and Drug Entrapment

### Insulin Loaded Nanoparticle Formulation Free Drug Percentage

The prepared insulin nanoparticle loaded formulations prepared were characterized for their free drug percentage. Different methodologies were used to calculate the percentage of free drug:
Method 1: The prepared nanoparticle dispersion was centrifuged at 10000 rpm for 1 hr at 2° C. The centrifugation was washed three times with 1 0 ml of HPLC water and further centrifuged 10000 rpm for 1 hr at 2° C.
Method 2: Frozen dried 100 mg of nanoparticle powder was dispersing in the 5 ml of the HPLC water. The dispersion was subjected to centrifugation Procedure at 1 0000 rpm for 1 5 min at 2° C. The supernatant was filtered using the 0.22μ membrane filter and analysed into the HPLC. The Percentage of free drug was determined using the following equation.

$$Percentage of free drug = \frac{Amount of drug in supernatant}{Initial amount} \times 100$$

### Determination of Encapsulate the Drug Quantity

The pellets were solubilized into 5 ml of 1% acetic acid/10 ml of water, vortexed 2 min and sonicate for 1 min. The sample was centrifuge at 15000 rpm for 30 minute at 2° C. The supernatant was filtered using the 0.22μ membrane filter and analyzed into the HPLC.

$$Percentage of encapsulated drug = \frac{Final amount of drug in supernantent}{Initial amount of the drug} \times 100$$

TABLE 3
Percentage free drug content and % of Entrapment drug

| | | | % of | % of |
|---|---|---|---|---|
| S | | Formulation | Free | Entrapment |
| No. | Method | code | drug | drug |
| 1 | Method 1 | N01 | Peak was | 2.04 |
| | | | not | |
| | | | identified | |
| 2 | | N1 | Peak was | — |
| | | | not | |
| | | | identified | |
| 3 | | N2 | Peak was | — |
| | | | not | |
| | | | identified | |
| 4 | | N3 | Peak was | — |
| | | | not | |
| | | | identified | |
| 5 | | N4 | Peak was | 2.38 |
| | | | not | |
| | | | identified | |
| 6 | | N5 | Peak was | 3.85 |
| | | | not | |
| | | | identified | |
| 7 | | N6 | 0.135 | 0.126 |
| 8 | | N7 | 4.611 | 0.277 |
| 9 | | N8 | 1.131 | 0.432 |
| 10 | | N9 | 6.898 | 0.801 |
| 11 | | N10 | 5.497 | 0.478 |
| 12 | | N11 | 6.420 | — |
| 13 | | N12 | 0.119 | — |
| 14 | | N13 | Not | Not |
| | | | Performed | Performed |
| 15 | | N14 | 1.783 | — |
| 16 | Method 2 | N15 | 47.80 | 66.45 |
| 17 | | N16 | 36.24 | 6.925 |
| 18 | | N17 A | 36.84 | 54.01 |
| 19 | | N17 B | 42.10 | 57.10 |
| 20 | | N17 C | 49.23 | 53.97 |
| 21 | | N18 | PLACEBO | |
| 22 | | N19 | PLACEBO | |
| 23 | | N20 | Formulation | |
| | | | not formed | |

Results: The percentage drug entrapment was found to be in a range of 2% to 66%. From the above table maximum results were obtained from the formulation prepared through Sodium Alginate, Chitosan and Calcium chloride i.e., N17A, N178, N17C.

### Particle Size, PDI and Zeta Potential

Procedure: For the purposes of determining the Zeta potential and particle size, the accurately measured 10 mg of the lyophilized powder was transferred to volumetric flask containing the 100 mm of HPLC water, vortex for 1 min and both sonicated for 1 min. The sample was further diluted 10 times with the water formulation was diluted 1000 times, vortex for 1 min and both sonicated for 1 min. The sample was transferred into the cuvettes and analyzed for the particle size and zeta potential. Observations are shown in Table 4 below.

TABLE 4
Particle size, PDI and Zeta potential of all prepared formulation

| | | | Poly | Zeta |
|---|---|---|---|---|
| S | Formulation | Particle | Dispersity | Potential |
| No. | code | size (nm) | Index (PDI) | (mV) |
| 1 | N01 | 5542 | 0.438 | — |
| 2 | N1 | Not performed | — | — |
| 3 | N2 | 255 | 0.180 | — |
| 4 | N3 | 895.5 | 0.355 | — |
| 5 | N4 | 502 | 0.381 | — |
| 6 | N5 | Not performed | — | — |
| 7 | N6 | 308.2 | 0.296 | — |
| 8 | N7 | 1037.6 | 0.427 | — |
| 9 | N8 | 429.3 | 0.251 | — |
| 10 | N9 | 8405 | 0.325 | — |
| 11 | N10 | 245.9 | 0.241 | — |
| 12 | N11 | Not performed | | |
| 13 | N12 | Not performed | | |
| 14 | N14 | Not performed | | |
| 15 | N15 | 539.6 | 0.304 | — |
| 16 | N16 | 432.7 | 0.273 | — |
| 17 | N17A | 434.6 | 0.443 | 31.4 |
| 18 | N17B | 384.0 | 0.021 | 24.4 |
| 19 | N17C | 346.2 | 0.228 | 34.5 |
| 20 | N18 | 445 | 0.341 | 25.6 |
| 21 | N19 | 661.89 | 0.757 | 43.4 |

Result and discussion: The particle size of all developed formulation was found to be in range of the 255 to 5542 nm. But in the case of the sodium alginate, chitosan polymer and calcium chloride crosslinker the particle size was found to be less than 500 nm. In addition to the particle size, the Poly dispersity Index (PDI) of the sodium alginate, chitosan polymer and calcium chloride formulation was found to be in a range of 0.2-0.4.

In case of the Eudragit polymer the particle size and PDI was found to be less than 850 nm and 0.2-0.4. Low PDI value represents the uniform sample with respect to particle size while increase in PDI value represents high polydispersity in sample with multiple particle size population.

Zeta potential obtained for the batches N17 A, N17 B, N17 C, is ranging between +24.4 mV to +34.5 mV. The positive zeta potential is due to the positive charges of the chitosan.

Based on the above finding the formulation code comprising the sodium alginate, chitosan and calcium chloride ie N17A for 100IU was selected and the same was formulation was prepared at low insulin strength 50IU and 25IU coded formulation N17B and N17C a was selected for the in vitro study.

### Example 4: Nanoparticle Formulation

Interaction studies Insulin Glargine loaded nanoparticles and excipients were performed using Fourier-transformed infrared spectroscopy for structure mapping. Accurately weighed amount 50 mg of the insulin loaded nanoparticles was transferred in the glass tube and 150 mg of each excipient was added in the vials separately. The vials were vortexed for 10 min to form a uniform mixture of the nanoparticle and excipients.

The sample for interaction studies comprised of Insulin nanoparticle+Guar gum (I: 3); Insulin nanoparticle+Magnesium stearate (I: 3); Insulin nanoparticle+HPMC KI00M (1:3); Insulin nanoparticle+Aerosil (1:3); Insulin nanoparticle+Avicel PH 101 (I: 3); Insulin nanoparticle+Guar gum+Magnesium stearate+HPMC K1 00M+Aerosil+Avicel PHI01. The IR spectra were recorded between 4000 cm·′ and 400 cm·′, with spectral resolution of 2 cm. Spectral analysis was performed using SPECTRUM software.

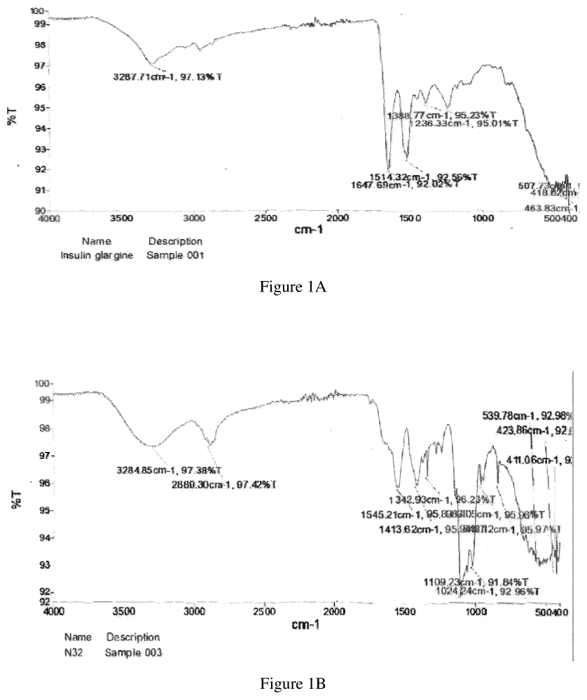

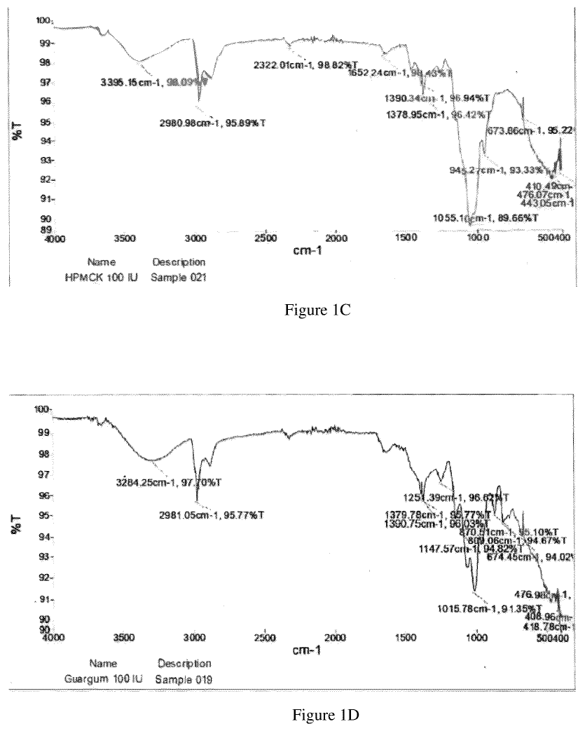

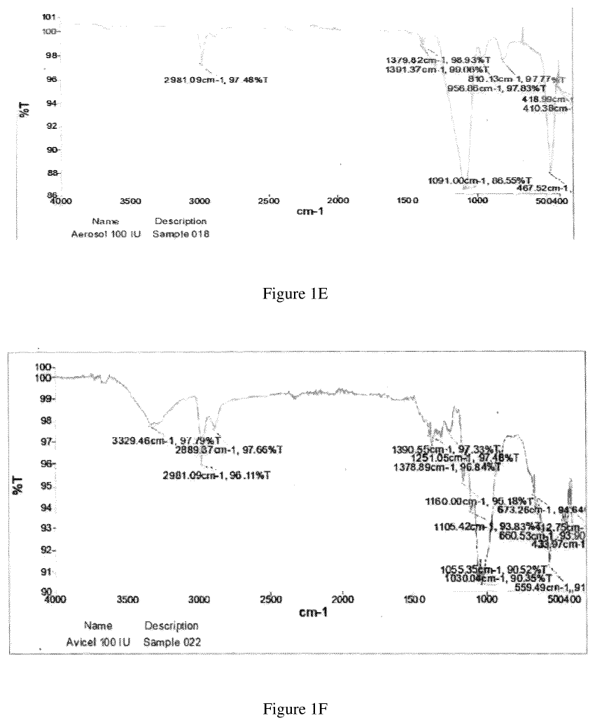

Results: The characteristics peaks of insulin glargine at 1647.69 and 1514.32 as shown in FIG. 1A was not observed when the FITR spectra of insulin loaded nanoparticle (FIG. 1B) and Spectra of insulin loaded nanoparticle and excipients (FIGS. 1C, 1D, 1E, 1F, 1G and 1H). Therefore, no interaction was observed between the insulin loaded nanoparticles and excipient using FTIR study.

### Example 5: Nanoparticle Formulation and Excipients Interaction Study

Interaction studies Insulin Glargine loaded nanoparticles and excipients were performed using differential scanning calorimeter for thermal stability measurements. Samples were prepared using same procedure as above stated experiment. Samples were scanned from 30° C. to 300° C., and scans were recorded at a rate of 10° C./min.

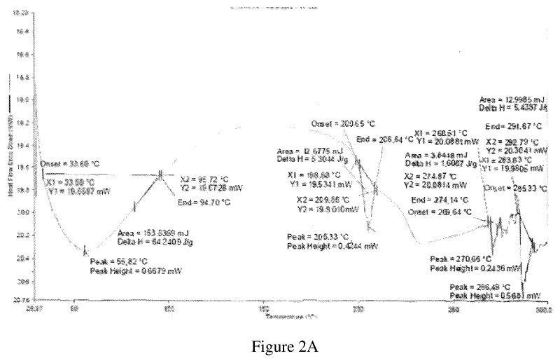

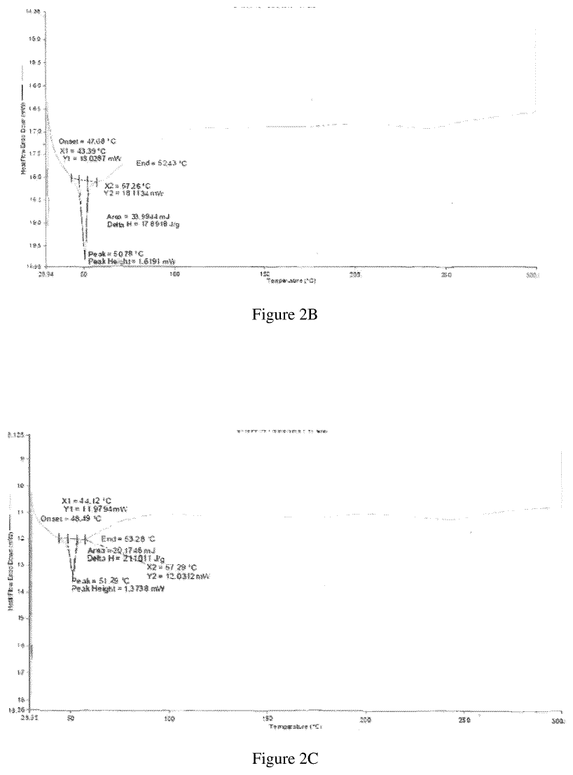

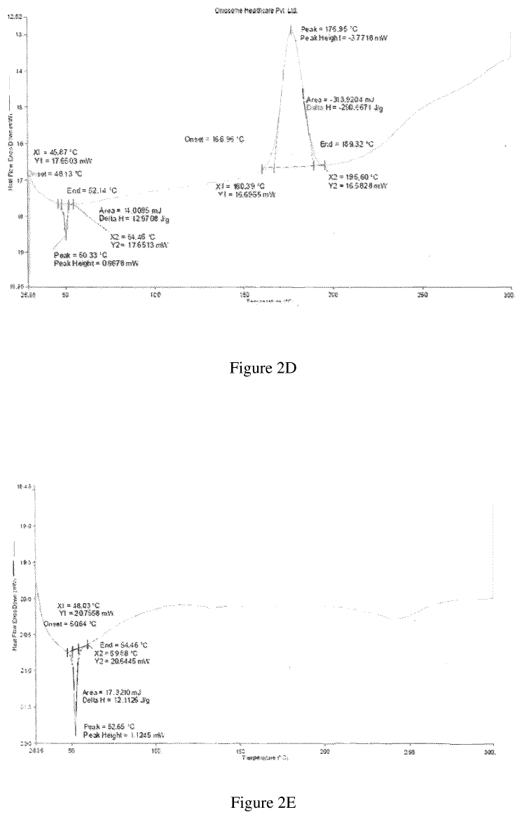

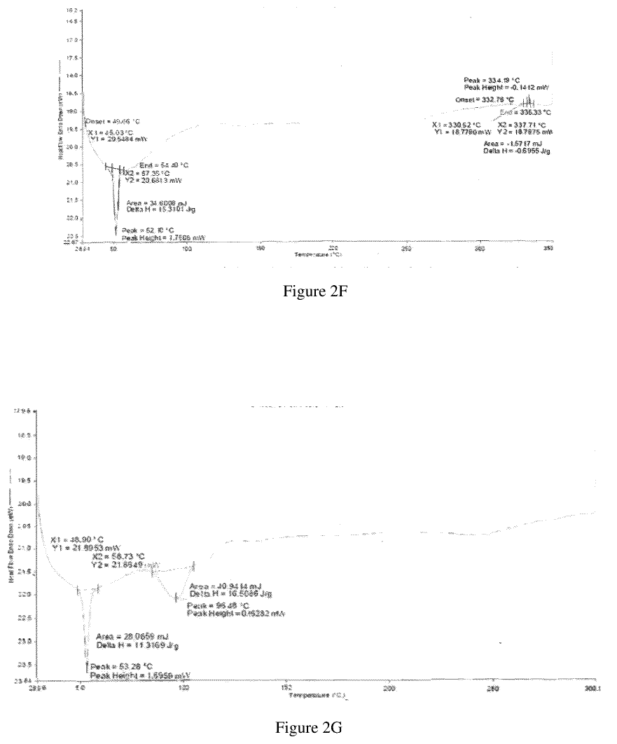

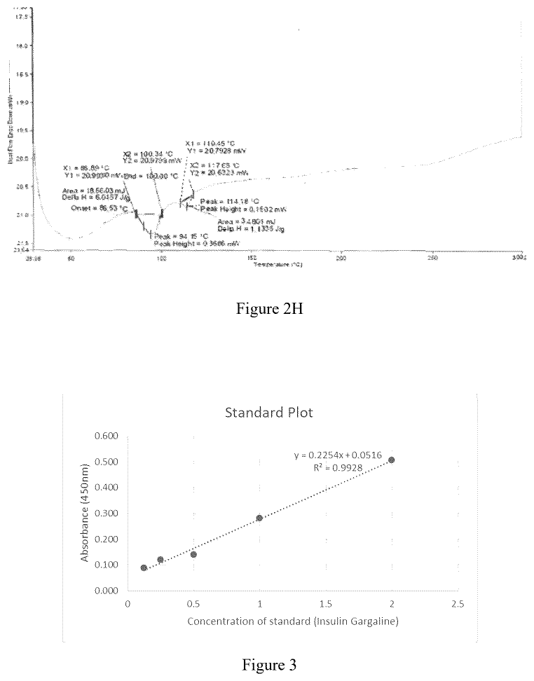

Results: DSC thermogram of insulin glargine demonstrated broad endothermic peak at 55.82° C., and one more endothermic peak at 205.33° C. as shown in FIG. 2A. Insulin loaded nanoparticle did not show the peak of the insulin glargine indicating the encapsulation of the insulin glargine in the nanoparticles (FIG. 2B, 2C). Further, DSC thermogram of the mixture of each excipient (FIGS. 2D, 2E, 2F, 2G and 2H) with the insulin glargine loaded nanoparticles did not show significant changes in the thermogram compared to the DSC thermogram of the nanoparticle. Thus indicate the no interaction was observed between the nanoparticle and excipients. Therefore, no interaction was observed between the insulin loaded nanoparticles and excipients using DSC study.

### Example 6: Cytotoxicity Analysis

CaCo-2 cell lines: Derived from Human Colon Adenocarcinoma as a popular representation of the intestinal epithelial barrier, the human epithelial cell line CaCo-2 has been employed. With brush boundary enzyme secreting microvilli growing on the apical side and regular tight connections forming between neighboring cells, CaCo-2 cells develop as a cylindrical polarised monolayer. The heterogeneous CaCo-2 cell line contains cells with somewhat varied characteristics. Therefore, the conditions of cultivation favor the expansion of certain cell subpopulations.

HeLa Cell Line: HeLa cell, a malignant cell from a strain that has been consistently grown since its isolation from a patient with cervical carcinoma in 1951. Henrietta Lacks' cancer was treated using the HeLa cell line, which is exceptionally resilient and prolific. Finite cell lines can only multiply as much as they can while immortal cell lines can replicate indefinitely. Telomerase is active in HeLa cells, which allows for unrestricted cell division and immortality. HeLa cells have contributed to some of the most important developments in a variety of disciplines, including cancer biology, infectious disease, basic microbiology, and many others.

MTT Assay is a colorimetric assay for measuring cellular proliferation, viability, and cytotoxicity in a nonradioactive manner. It is predicated on the capacity of cellular oxidoreductase enzymes to convert the purple formazan, an insoluble form of the tetrazolium dye MTT, to NADPH-dependent levels. Following solubilization, the amount of formazan formed may be measured spectrophotometrically (at 570 nm) and is inversely correlated with the viability of the cultured cells as per OEDC No. 129 Guidance Document on Using Cytotoxicity Tests to Estimate Starting Doses for Acute Oral Systemic Toxicity Tests and OECD No. 491 Guideline for Testing of Chemicals: Short Time Exposure In Vitro Test Method for Eye Hazard Potential.

Preparation of Tester Cells: CaCo-2 and HeLa cells were cultured in tissue grade T25 flask with respective media i.e. MEM+20% FBS (Caco-2) and RPMI+10% FBS (HeLa) and incubated at 37° C. in CO2 incubator for 72 hrs. After attaining 90% confluency, the cells were transferred to tissue grade T75 flask and incubated at 37° C. in CO2 incubator. Further after attaining 90% confluency in T75, the cells were harvested, and cell counting was performed by trypan blue staining.

Control: An equal volume of respective fresh media i.e. MEM+20% FBS (CaCo-2) and RPMI+10% FBS (HeLa) containing cells were used as control.

Blank: An equal volume of respective fresh media i.e. MEM+20% FBS (CaCo-2) and RPMI+10% FBS (HeLa) were used as blank.

Test Article Preparation: The test articles were subjected to the preparation under sterile conditions, stock solution (2 mg/ml, as mentioned by the sponsor) was used to prepare serial dilutions of each sample in DPBS buffer at the concentration of 1 mg/ml, 0.5 mg/ml, 0.25 mg/ml and 0.125 mg/ml.

Standard: Equal volume of Insulin glargine (provided by the sponsor) was used as standard by preparing serial dilutions in DBPS buffer at similar concentrations as that of test articles (2 mg/ml, 1 mg/ml, 0.5 mg/ml, 0.25 mg/ml and 0.125 mg/ml).

### Test Procedure

Cell Seeding and Trypsinization: 5.0 ml Trypsin was added to T75 flasks having 90% confluency of CaCo-2- and HeLa and incubated for 10.0 mins. After 10.0 mins, when trypsin had turned turbid, it was collected in falcons with 15.0 ml fresh complete growth media i.e. MEM+20% FBS (Caco-2) and RPMI+10% FBS (HeLa) and centrifuged at 400 g for 5.0 mins. Obtained supernatant was discarded and the pellet containing cells were diluted in 1 ml of respective fresh complete growth media i.e. MEM+20% FBS (Caco-2) and RPMI+10% FBS (HeLa). 50.0 μl of media containing cells were added to 50.0 μl of Trypan blue (1:1) and loaded on hemocytometer for cell counting. Homogenous mixtures of 1×106 cells in 10 ml media were prepared and around 1×104 cells per well were seeded in 96 well plates in their respective complete growth media i.e. MEM+20% FBS (Caco-2) and RPMI+10% FBS (HeLa).

Cell Proliferation: After the seeding of cells, 96 well plates containing 1×104 cells with 200 ul of media per well were incubated overnight at 37° C. in CO2 incubator. Next day, the cells were treated with the different concentrations of each test article (2 mg/ml, 1 mg/ml, 0.5 mg/ml, 0.25 mg/ml and 0.125 mg/ml) followed by incubation at 37° C. in CO2 incubator.

MTT Assay: After 24 hours of incubation, old media was removed from the wells and fresh media along with MTT solution was added to each well and incubated for 4 hours. Formed formazan crystals were then solubilized by adding DMSO to each well and incubated for 2 hours. After incubation, absorbance was recorded at 570 nm using SpectraMax iD3

Results: The absorbance data of each well was subtracted with blank well readings. The percentage (%) viability of each concentration of product was calculated by comparing with untreated (control) wells and have been provided in the below Table 5.

TABLE 5
% Cell Viability of CaCo-2 and HeLa cells with tested items w.r.t standard.

| | Blank | Control | 2 mg/ml | 1 mg/ml | 0.5 mg/ml | 0.25 mg/ml | 0.125 mg/ml |
|---|---|---|---|---|---|---|---|
| | Insulin Glargine | | | | | | |
| CaCo-2 | 0 | 100 | 53.92* | 56.68* | 59.23* | 64.31** | 72.5** |
| HeLa | 0 | 100 | 60.81 | 64.64* | 67.94* | 73.03* | 85.21** |
| | N17A | | | | | | |
| CaCo-2 | 0 | 100 | 72.92 | 73.4 | 79.48* | 81.77** | 84.58** |
| HeLa | 0 | 100 | 74.3 | 77.07 | 81.38* | 86.02** | 92.06** |
| | N17B | | | | | | |
| CaCo-2 | 0 | 100 | 61.81 | 64.32 | 71.21* | 75.13* | 77.59* |
| HeLa | 0 | 100 | 69.46 | 71.32 | 77.27* | 86.52* | 98.41** |
| | N17C | | | | | | |
| CaCo-2 | 0 | 100 | 66.35 | 69.46 | 70.47* | 71.25* | 74.96* |
| HeLa | 0 | 100 | 68.84 | 70.37 | 76.46* | 83.01** | 91.49** |
| | N18 Placebo | | | | | | |
| CaCo-2 | 0 | 100 | 66.71 | 74.36* | 78.42* | 80.23** | 84.76** |
| HeLa | 0 | 100 | 70.52 | 73.85 | 85.38* | 89.49** | 90.26** |
| | N19 Placebo | | | | | | |
| CaCo-2 | 0 | 100 | 75.73 | 76.51 | 77.88* | 80.3* | 85.12* |
| HeLa | 0 | 100 | 75.2 | 78.48 | 86.21* | 89.39* | 92.06* |

*Showed significant association of viability of cells with respect to the control;
**Showed highly significant association of viability of cells with respect to the control.

From the above results it is concluded that during the in-vitro cytotoxicity study of test articles “N17 A, N17 B, N17 C, N18 Placebo, and N19 Placebo, cell viability of Caco-2 and HeLa increased with increase in dilutions from 2 mg/ml to 0.125 mg/ml of the test article as compared to the control. However no significant decrease in viability of CaCo-2- and HeLa cells were observed in all the concentrations of the test articles, implying the test articles to be non-cytotoxic and is safe for use.

EXAMPLE 6: In-Vitro study to evaluate the effect of test materials “N17 A to N17 C” on the uptake of insulin by CaCo-2 cells by Enzyme Linked Immunosorbent Assay (ELISA).

Preparation of Tester Cells: CaCo-2 cells were cultured in tissue grade T25 flask with respective media i.e. MEM+20% FBS (Caco-2) and incubated at 37° C. in CO2 incubator for 72 hrs. After attaining 90% confluency, the cells were transferred to tissue grade T75 flask and incubated at 37° C. in CO2 incubator. Further after attaining 90% confluency in T75, the cells were harvested, and cell counting was performed by trypan blue staining.

Control: An equal volume of fresh media (MEM+20% FBS) containing cells was used as control.

Blank: An equal volume of fresh media (MEM+20% FBS) was used as blank.

Test Article Preparation: The test articles were subjected to the preparation under sterile conditions, stock solution (2 mg/ml, as mentioned by the sponsor) was used to prepare serial dilutions of each sample in DPBS buffer at the concentration of 2 mg/ml, 1 mg/ml, 0.5 mg/ml, 0.25 mg/ml and 0.125 mg/ml.

Standard: Equal volume of Insulin glargine (provided by the sponsor) was used as standard by preparing serial dilutions in DBPS buffer at similar concentrations as that of test articles (2 mg/ml, 1 mg/ml, 0.5 mg/ml, 0.25 mg/ml and 0.125 mg/ml).

Insulin load: The total insulin load was given to each well in 6-well plate with a concentration of 2747 IU/well.

### Test Procedure

Cell Seeding and Trypsinization: 5.0 ml Trypsin was added to T75 flasks having 90% confluency of CaCo-2 and incubated for 10.0 mins. After 10.0 mins, when trypsin had turned turbid, it was collected in falcons with 15.0 ml fresh complete growth media (MEM+20% FBS) and centrifuged at 400 g for 5.0 mins. Obtained supernatant was discarded and the pellet containing cells were diluted in 1 ml of respective fresh complete growth media (MEM+20% FBS). 50.0 μl of media containing cells were added to 50.0 μl of Trypan blue (1:1) and loaded on hemocytometer for cell counting. Homogenous mixtures of 50×106 cells in 50 ml media were prepared and around 1×106 cells per well were seeded in 6 well plates in complete growth media (MEM+20% FBS). Cells were calculated by using Neubauer chamber.

Cell Proliferation: After the seeding of cells, 96 well plates containing 1×104 cells (Cells were calculated by using Neubauer chamber) counting with 200 ul of media per well were incubated overnight at 37° C. in CO2 incubator. Next day, the cells were loaded with 2747 IU of insulin glargine and incubated for 24 hours at 37° C. in CO2 incubator. The cells were treated with 100 μl of different concentrations of each test article and Insulin Glargine (2 mg/ml, 1 mg/ml, 0.5 mg/ml, 0.25 mg/ml and 0.125 mg/ml) followed by incubation for 24 hours at 37° C. in CO2 incubator. Next day the cells were collected from 6 well plates and were centrifuged at 600 g to obtain the cell lysates and separate the secretome for further analysis.

ELISA: Cell lysates were then suspended in 100 μl of DPBS and were then subjected to Human Insulin ELISA kit (Cat. No.-RAB0327, Sigma Aldrich) protocol for the detection of insulin uptake. Absorbance was then observed at 450 nm using SpectraMax iD3.

The plate was neutralized to 0.00000 for blank and the absorbance data of each well was subtracted with blank well readings (Culture media only). The standard plot were prepared as per the absorbance (450 nm) and the Insulin Glargine concentration as shown in the FIG. 3.

On the basis of the absorbance in the Insulin Glargine the percentage uptake of insulin by the cells (CaCo-2) has been calculated as shown in Table 6.

TABLE 6
Percentage uptake of Insulin Glargine dilutions

| | |
|---|---|
| Dilutions | Percentage |
| (Insulin Glargine) | Uptake |

| | | |
|---|---|---|
| 2 | mg/ml | 58.437 |
| 1 | mg/ml | 23.667 |
| 0.5 | mg/ml | 1.752 |
| 0.25 | mg/ml | No Uptake |
| 0.125 | mg/ml | No Uptake |

Furthermore, the insulin uptake was calculated for each test item at tested concentration in the CaCo-2 cells and has been shown in Table 7.

TABLE 7
Percentage uptake of insulin by cells treated in lysate and
the secretome with test compounds at the concentration of
2 mg/ml, 1 mg/ml, 0.5 mg/ml, 0.25 mg/ml and 0.125 mg/ml.

| | | Dilutions | | | | |
|---|---|---|---|---|---|---|
| | | 2 mg/ml | 1 mg/ml | 0.5 mg/ml | 0.25 mg/ml | 0.125 mg/ml |
| | | Percentage Uptake | | | | |
| Test | Insulin | 58.437 | 23.667 | 1.752 | No Uptake | No Uptake |
| Compound | Glargine | | | | | |
| | Control | 41.008 | | | | |
| | N17 A | 80.383 | 72.272 | 64.583 | 58.668 | 48.106 |
| | N17 B | 64.583 | 60.189 | 55.119 | 49.458 | 46.416 |
| | N17 C | 57.739 | 53.007 | 50.134 | 47.768 | 45.149 |
| | N 18 | 40.84 | 41.093 | 41.093 | 40.84 | 41.347 |
| | PLACEBO | | | | | |
| | N 19 | 41.093 | 41.178 | 41.262 | 41.009 | 41.009 |
| | PLACEBO | | | | | |

TABLE 8

| | | Dilutions | | | | |
|---|---|---|---|---|---|---|
| | | 2 mg/ml | 1 mg/ml | 0.5 mg/ml | 0.25 mg/ml | 0.125 mg/ml |
| | | Percentage in the secretome | | | | |
| Test | Insulin | 41.534 | 77.210 | 98.186 | 99.764 | 99.837 |
| Compound | Glargine | | | | | |
| | Control | 41.008 | | | | |
| | N17 A | 19.611 | 25.521 | 35.409 | 41.305 | 51.864 |
| | N17 B | 35.415 | 39.781 | 44.851 | 50.242 | 53.534 |
| | N17 C | 42.221 | 46.873 | 49.842 | 52.182 | 54.822 |
| | N 18 | 59.143 | 58.757 | 58.856 | 59.095 | 58.543 |
| | PLACEBO | | | | | |
| | N 19 | 58.706 | 58.764 | 58.456 | 58.86 | 58.851 |
| | PLACEBO | | | | | |

As per the given details by the sponsor the insulin uptake was compared with the N18 and N19 placebo compounds and the significant value have been calculated by paired t-test using SPSS software (ver. 16.0). The data shown in Table 9 below.

TABLE 9
Percentage uptake of insulin by cells treated with test compounds w.r.t. N18 Placebo

| | | Dilutions | | | | | P- | |
|---|---|---|---|---|---|---|---|---|
| | | 2 mg/ml | 1 mg/ml | 0.5 mg/ml | 0.25 mg/ml | 0.125 mg/ml | value^{a} | P- |
| | | Percentage Uptake w.r.t. N 18 Placebo | | | | | | value^{b} |
| Test | N17 A | 196.826 | 175.873 | 157.162 | 143.655 | 116.349 | 0.003 | <0.000 |
| Compound | N17 B | 158.137 | 146.470 | 134.132 | 121.103 | 112.261 | 0.003 | <0.000 |
| | N17 C | 141.379 | 128.992 | 122.001 | 116.965 | 109.196 | 0.002 | <0.000 |

^{a}Calculated P-value with respect to Placebo;
^{b}Calculated P-value with respect to Insulin Glargine.

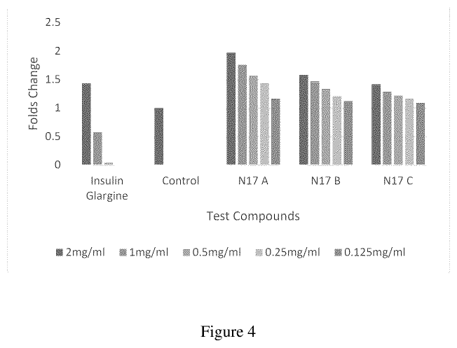

From the results (see FIG. 4) it is concluded that during the in-vitro study of the insulin uptake by the CaCo-2 cells by the tested compounds at variable concentrations of test articles “N17 A, N17 B, N17 C, N18 Placebo and N19 Placebo”. It was observed that the placebo compounds i.e. N18 placebo and N19 placebo showed similar results with that of control, means no insulin was loaded in these compounds and makes the results authenticity. It was observed that the N17A showed almost 2 folds of uptake at the concentration of 2 mg/ml while the trend was observed with the decrease in the dilution in the tested compounds. To make the confirmation of uptake the secretome insulin were also measured and the percentage was found to be constant to make the certainty of results. However, all the tested compounds showed significant insulin uptake when compared with the placebo test items viz. N 18 and N 19 (p≤0.005) and highly significant as compared with the Insulin Glargine. It was also evident from the results that the test compounds are safe for use in human cells. Results and conclusion apply only to the test articles tested i.e., N17 A, N17 B, N17 C, N18 Placebo and N19 Placebo.

The foregoing description of the invention has been set merely to illustrate the invention and is not intended to be limiting. Since the modifications of the disclosed embodiments incorporating the spirit and substance of the invention may occur to the person skilled in the art, the invention should be construed to include everything within the scope of the disclosure.

1. An oral formulation comprising:
a. peptides or biosimilar;
b. alginates or an alkali salt thereof;
c. at least one polymer;
d. crosslinker; and
one or more pharmaceutically acceptable excipients,
wherein at least one polymer is selected from arabinoxylans, gelatin, pectin, guar gum, hyaluronic acid, mucin, poly-amino acids, chitosan, dextran, PEG, galactomannan, starch;
wherein the chitosan has a molecular weight range of 20 kda to 2000 kda.

2. The formulation as claimed in claim 1, wherein the formulation is a nanoparticle.

3. The formulation as claimed in claim 1, wherein the peptide is selected from insulin, preferably human insulin, Long-acting, Short-acting, Rapid-acting, Intermediate-acting and Mixed Insulin.

4. The formulation as claimed in claim 1, wherein the insulin is insulin glargine.

5. The formulation as claimed in claim 1, wherein the alginates or an alkali salt thereof is selected from sodium alginate.

6. (canceled)

7. The formulation as claimed in claim 1, wherein the chitosan is more than 85% deacylated chitosan having molecular weight in the range of 190 Daltons to 310 Daltons.

8. The formulation as claimed in claim 1, wherein the crosslinker is selected from calcium chloride, calcium salicylate, collagen, lignin, carbodiimides or combination thereof.

9. The formulation as claimed in claim 1, wherein the oral formulation is a nanoparticle encapsulating insulin glargine.

10. The formulation as claimed in claim 1, wherein the peptide is in the range of 0.1 to 60%.

11. The formulation as claimed in claim 1, wherein the insulin is in the range of 1 IU to 200 IU, or 20 IU to 110 IU.

12. The formulation as claimed in claim 1, wherein the alginic acid or an alkali salt thereof is in the range of 0.1 wt % to 50 wt %, or 1 wt % to 2 wt %.

13. The formulation as claimed in claim 1, wherein the crosslinker is in the range of 0.01% to 10%.

14. The formulation as claimed in claim 1, wherein the pharmaceutically acceptable excipients are selected from polyvinyl alcohol, polaxmer188, glycerol or derivative thereof, fatty acids, ethylene oxide, alcohol, bile salts, phospholipids or combination thereof.

15. The formulation as claimed in claim 1, optionally comprising at least one drug.

16. The formulation as claimed in claim 1, selected from pill, powder, capsule, or nano-emulsion.

17. A process for preparing an oral formulation comprising the following steps:
preparing insulin solution of different strength in water by adjusting the 4.0 pH,
adding insulin solution to 1% sodium alginate,
adding chitosan in acetic acid to an aqueous solution mixing with continuous homogenizing the solution at 12000 rpm at 2-8° C. for 10 minutes to prepare a first homogenized mixture;
optionally adding an aqueous solution containing a mixture of pharmaceutical excipients to the first homogenized mixture containing insulin and mixing to prepare a second homogenized mixture;
sonicating the second homogenize mixture for 3 mins in an ice bath followed by centrifuging for 60 mins at 10,000 rpm, washing only one time and centrifuging again for 5 mins to prepare the nanoparticle pellets;
freeze drying the pellet to obtain the oral formulation.

18. The process as claimed in claim 17, wherein the chitosan in acetic acid is in the ratio of 1:1.

19. The process as claimed in claim 17, wherein the sodium alginate in the first homogenized mixture is in the range 0.2% to 10%.

20. The process as claimed in claim 17, wherein the insulin solution is in the range 0.1% to 50%.

21. The process as claimed in claim 17, wherein the first homogenized mixture was prepared by mixing at 5000 to 15000 rpm, and the second homogenized mixture was prepared by mixing at 5000 to 15000 rpm.

22. The process as claimed in claim 17, wherein the first homogenized mixture was mixed for 5 to 20 minutes, the second homogenized mixture was mixed for 2 to 10 minutes, and the second homogenized mixture was centrifuged for 60 min at 10,000 rpm and at 2-8° C. temperature.

23. The process for preparing an oral formulation as claimed in claim 17, comprising the following steps:
adding chitosan (2% w/v) in acetic acid (1% v/v) to 20 ml of Sodium Alginate as the aqueous phase and homogenizing the mixture for 10 min at 12,000 rpm;
adding 5 ml of insulin solution (100 IU/ml) to the mixture and mixing it gently using a magnetic stirrer for 3 min at 200 rpm;
adding an aqueous phase containing a mixture of 5 ml of Poloxamer-188 2 and 5 ml of PVA to the phase containing the insulin and chitosan and homogenizing for 5 min at 12000 rpm;
sonicating the preparation at 80 W for 2 min in an ice bath followed by centrifuging (10,000 rpm, 10° C., 60 min) and after two washing it was centrifuged again at 10,000 rpm for 15 min;
freeze-drying to obtain encapsulated Insulin glargine.

24. A method for treatment of diseases by administering the oral formulation as claimed in claim 1.

25. The method as claimed in claim 24, wherein the oral formulation is administered for decreasing blood glucose or for treating conditions related to elevated blood glucose in a human comprising administering the oral formulation as claimed in claim 1, wherein the condition related to elevated blood glucose is selected from the group consisting of Type 1 diabetes, Type 2 diabetes, impaired glucose tolerance, hyperglycemia, insulin resistance syndrome, and glucosuria.

26. The method as claimed in claim 24, wherein the oral formulation is administered for delivering peptides to the gastrointestinal tract, including insulin at the ileo-caecal junction of colon.

27. The method as claimed in claim 24, wherein the peptides are selected from Bortezomib, Cosyntropin, Saralasin, Vancomycin, Icatibant, Salmon calcitonin, Pentagastrin, Corticorelin, Cyclosporin, Urofollitropin, Glucagon, Exenatide, Teduglutide, Eptifibatide, Buserelin, Enfuvirtide, Somatorelin, Linaclotide, Romidepsin, Vapreotide, Ziconotide, Mifamurtide, Carbetocin, Teriparatide, Secretin (human), Somatostatin, Bortezomib, Cosyntropin, 26S proteasome Bortezomib ACTH er Cosyntropin Angiotensin II, Bradykinin B2, Corticorelin or combination thereof.

28. The formulation as claimed in claim 1, for use in the treatment of diseases selected from cortisol disorder, Anti-hypertension, Hereditary angioedema, osteoporosis, immunosuppressant, fertility treatment, irritable bowel syndrome, myocardial infarction, Sex hormone-responsive cancers, anti-HIV, hormone deficiency, irritable bowel syndrome, esophageal variceal bleeding, chronic pain, osteosarcoma, postpartum bleeding, osteoporosis, multiple myeloma or prostate cancer.

29. The formulation as claimed in claim 1, for use in delivering diagnostic aids/agents.
